(19) United States
(12) Patent Application Publication
(10) Pub. No.: US 2018/0028690 A1
(43) Pub. Date: Feb. 1, 2018

(54) INDOCYANINE GREEN FORMULATIONS AND METHODS FOR IMAGING OF THE URINARY PATHWAYS

(71) Applicants: Yissum Reseach Development Company of the Herbrew University of Jerusalem Ltd., Jerusalem (IL); The Medical Research, Infrastructure, and Health Services Fund of the Tel-Aviv Medical Center, Tel Aviv (IL)

(72) Inventors: Eran NIZRI, Jerusalem (IL); Shlomo MAGDASSI, Jerusalem (IL); Emma PORTNOY, Jerusalem (IL); Sara EYAL, Jerusalem (IL)

(21) Appl. No.: 15/550,283

(22) PCT Filed: Feb. 11, 2016
(86) PCT No.: PCT/IL2016/050162

§ 371 Date: Aug. 10, 2017

(30) Foreign Application Priority Data
Feb. 12, 2015 (IL) 237210

(51) Int. Cl. A61K 49/00

(52) U.S. Cl. CPC A61K 49/0034; A61K 49/0039; A61K 49/0043

(57) ABSTRACT

The present invention relates to an imaging composition for imaging the urinary pathways, which comprises particles each independently comprising (a) a phospholipid, wherein a near infrared (NIR) fluorescent probe is non-covalently linked to said particle; or (b) an inclusion complex of said NIR fluorescent probe and either an hydroxyalkyl- or sulphoalkylether-cyclodextrin; and to a method of use.

### TECHNICAL FIELD

The present invention relates to an imaging composition for imaging the urinary pathways, and to a method of use.

### BACKGROUND ART

Iatrogenic ureteral injury is a serious complication of abdominal surgeries, with a reported incidence of up to 10% (Summerton et al., 2012; Vakili et al., 2005). Unfortunately, this incidence is on the rise as more procedures are performed laparoscopically (Assimos et al., 1994). Recognition of such injury is often delayed, which further complicates the outcome (Meirow et al., 1994). Treatment requires endoscopic interventions and/or surgical reconstructive procedures, which may be associated with further complications such as ureteral strictures and recurrent urinary tract infections (Summerton et al., 2012; Redan and McCarus, 2009; Sorinola and Begum, 2005). Accordingly, ureteral injury is associated with reduced quality of life, increased medical costs and legal litigation. Hence, intraoperative identification and prevention of ureteral injury are vital.

The common method for intraoperative identification of the ureters is based on retroperitoneal dissection and direct ureteral detection with confirmation of peristalsis. Insertion of ureteral catheters with cystoscopy by an urologist may be helpful when their need is anticipated, although this insertion entails additional procedure by a skilled provider (Larach and Gallagher, 2000). Imaging technologies, such as preoperative intravenous pyelography, retrograde pyelography, or urologic computed tomography can also prevent injury. However, these procedures themselves can lead to additional complications instead of simplifying the surgery and they do not provide real-time guidance during the actual operation (Polom et al., 2014). Optical imaging using near-infrared (NIR) fluorescent light has the potential to address these issues. Compared to other optical imaging methods, NIR is characterized by relatively deep tissue penetration, low toxicity and high signal to noise ratio, due to the low autofluorescence of biological molecule at that range of wavelengths (Luo et al., 2011). Moreover, the combination of a specialized camera and NIR fluorophores can illuminate the ureters without altering the surgical field, because at low concentrations these dyes are invisible to the human eye (Troyan et al., 2009).

Up-to-date, the only United States Food and Drug Administration (FDA) and European Medicines Agency (EMA) human approved NIR molecule is indocyanine green (ICG), a water soluble tricarbocyanine dye. ICG is widely used in the clinic for determination of cardiac output, hepatic function and liver blood flow, inspection of retinal and choroidal vessels (Dzurinko et al., 2004), and diagnosis of burn depth (Still et al., 2001). Unfortunately, ICG's elimination is mostly hepatic, through a variety of uptake and efflux transporters (Bax et al., 1980; Huang and Vore, 2001; Portnoy et al., 2012), with negligible non-hepatic elimination (Bax et al., 1980; Cherrick et al., 1960). Attempts to enhance the NIR visualization of the urinary tract involved the use of a novel fluorophore CW800-CA in pigs, but it was not advantageous to ICG (Tanaka et al., 2007).

Liposomes are a very attractive delivery form because they are physically and chemically well-characterized structures that can be delivered through almost all routes of administration, and are biocompatible (Barenholz and Crommelin, 1994). Utilization of ICG-loaded liposomes in biological systems was recently described by Sandanaraj et al. (2010) and Proulx et al. (2010).

International Publication No. WO 2012/032524, herewith incorporated by reference in its entirety as if fully disclosed herein, discloses a liposomal formulation for detection of tumors in the gastrointestinal track, wherein at least one NIR fluorescent probe such as ICG and at least one active agent, e.g., a peptide, polypeptide or protein, are non-covalently bound to the outer surface of phospholipid-based particles, i.e., passively adsorbed to said phospholipid-based particles.

Cyclodextrins, more specifically 2-hydroxypropyl-β-cyclodextrin (HPβCD) and sulphobutylether-β-cyclodextrin (Captisol®), are FDA approved for various administration routes, and are known to be eliminated mostly by renal excretion (Stella and He, 2008; Gould and Scott, 2005). As recently found, ICG forms complexes with cyclodextrins, which stabilize it (Barros et al., 2010).

### SUMMARY OF INVENTION

It has now been found, in accordance with the present invention, that the emission intensity of ICG from ureters, when it is non-covalently linked to phospholipid-based particles such as liposomes and micelles, i.e., adsorbed to, embedded within or encapsulated within said particles, or alternatively formulated as cyclodextrin-based inclusion complexes, is unexpectedly significantly increased compared to that of free ICG.

In one aspect, the present invention thus relates to a method for imaging the urinary pathways of an individual in need thereof, said method comprising:

(i) systemically administering to said individual an imaging composition comprising particles each independently comprising (a) a phospholipid, wherein a NIR fluorescent probe is non-covalently bound to said particle, i.e., either adsorbed to or embedded within said particle; or (b) an inclusion complex of said NIR fluorescent probe and either an hydroxyalkyl- or sulphoalkylether-cyclodextrin; and

(ii) quantitatively or qualitatively measuring the emission intensity of said NIR fluorescent probe from the urinary pathways of said individual upon excitation at a proper wavelength, thereby imaging said urinary pathways.

In one particular such aspect, the method of the present invention is used for imaging, more particularly single-shot or repetitive imaging, the urinary pathways of said individual during a surgery such as an abdominal or pelvic surgery, e.g., for intraoperative identification or prevention of iatrogenic ureteral injury.

In certain embodiments, the imaging composition administered according to the method of the present invention comprises particles each independently comprising (a) a lecithin, e.g., Phospholipon® 50 or Phospholipon® 75 (mainly composed of a phospholipid mixture), wherein a cyanine dye such as ICG is either adsorbed to or embedded within said particle; or (b) an inclusion complex of said cyanine dye and either hydroxyalkyl-β-cyclodextrin, e.g., HPβCD, or sulphoalkylether-β-cyclodextrin, e.g., Captisol®.

In another aspect, the present invention provides an imaging composition as defined above, i.e., an imaging composition comprising particles each independently comprising (a) a phospholipid, wherein a NIR fluorescent probe is non-covalently bound to said particle, i.e., either adsorbed to or embedded within said particle; or (b) an inclusion complex of said NIR fluorescent probe and either an hydroxyalkyl- or sulphoalkylether-cyclodextrin, for use in imaging the urinary pathways.

In a further aspect, the present invention relates to use of an imaging composition as defined above for the preparation of a medicament for imaging the urinary pathways.

### BRIEF DESCRIPTION OF DRAWINGS

FIG. 1 shows a representative in vivo NIR image of female mice treated with liposomal vs. free ICG. Image was acquired approximately 20 min after intravenous injection of 8 mg/kg ICG in liposomes or as a solution in sucrose buffer. The urinary bladder is indicated by arrows. Also indicated is the liver (L) and gallbladder (G).

FIGS. 2A-2B show ICG fluorescence in mice treated with liposomal vs. free ICG: Area under the concentration-time curve (AUC) of ICG emission in feet, liver, gallbladder and urinary bladder (2A); and feet-normalized AUC of gallbladder, liver and the urinary bladder (2B). n=5 mice per treatment. *Significantly different from gallbladder intensity in liposomes-treated and in free ICG-treated mice, P<0.05.

FIG. 3 shows distribution of liposomal and free ICG in mouse tissues. Tissues were collected from female mice 10 min after intravenous injection of 8 mg/kg ICG in liposomes or as a solution in sucrose buffer. n=6 mice per treatment. *Significantly different from intensity in ureters of ICG-treated mice, P<0.05.

FIG. 4 shows a representative NIR image of the urinary pathways in female mice intravenously treated with free (panels A and B) or liposomal (panels C and D) ICG (8 mg/kg ICG). The mice were then subjected to midline laparotomy, the uterus was removed and the ureters were clipped or loosely tied for identification. The ureters are indicated by arrows. Also shown in panel A are the kidneys (K) and the liver (L).

FIGS. 5A-5D show in vivo renal and ureteral distribution of liposomal and free ICG: ICG tissue emission intensity in ureters and kidneys (5A); ureters/kidneys ratios of emission intensity (5B); correlation between ureteral and renal emission intensity (correlation analyses were performed separately for liposomal ICG- and free ICG-treated mice) (5C); and correlation between kidney/blood ratio of emission intensity and blood emission intensity (5D). Represented are all animals for which blood samples were available. For 5A and 5B, the numbers of mice within each group are indicated on the bars. *Significantly different from values in ICG-treated mice, P<0.01.

FIGS. 6A-6C show ICG localization within liposomes. 6A and 6B show ICG interactions with pyrene, wherein 6A shows pyrene emission intensity ratios I_{I}/I_{III} as a function of ICG concentration (pyrene concentration was 2×10⁻⁵ M); and 6B shows pyrene emission intensity ratios I_{E}/I_{M} as a function of ICG concentration (pyrene concentration was 2×10⁻⁴ M). ICG concentration range was 0-8.0×10⁻⁵ and Phospholipon® 50 liposomes concentration was 1% (w/w) (n=5). 6C shows ICG effect on DMPC thermotropic behavior. DSC thermograms of DMPC liposomes as a function of temperature. DMPC concentration was 4.4% (w/w) and ICG concentrations were 0-1.5×10⁻³ M (n=4).

FIGS. 7A-7B show the absorbance spectrum of ICG in solution (5 mM phosphate buffer; pH 7; 7A) and in Phospholipon® 50 liposomes (7B), demonstrating the effect of liposomes on ICG absorbance spectra.

FIG. 8 shows a representative experiment indicating that ICG-HPβCD complex is renally excreted whereas free ICG is negligibly excreted in the urine. FVB mice were injected with free ICG (panel A) or ICG-HPβCD complex (panel B). The black and white arrows show the kidney and the ureter, respectively.

FIG. 9 shows the quantification of ICG-HPβCD complex brightness intensity in the ureter, as compared to the retroperitoneum as a reference tissue. The results summarize 3 different experiments (n=6 for each group).

FIG. 10 shows liposome size as a function of sonication time. Results are from one representative experiment out of 7.

FIG. 11 shows cryo-TEM picture of the liposomes generated by method described in Study 3.

FIGS. 12A-12B show size distribution of the liposomes produced by the method described in Study 3, after 40 min (12A) and 20 min (12B) of sonication.

FIG. 13 shows liposomal solution stability. Maximal fluorescence intensity was determined at different time points after preservation of the liposomes in light protected and cooled (4° C.) environment.

FIG. 14 shows in vivo ureter/retroperitoneum imaging with liposomes of different liposomal size, demonstrating that the best ureteral visualization was achieved by liposomal size of 30-60 nm.

### DETAILED DESCRIPTION OF THE INVENTION

The present invention provides a method for imaging the urinary pathways by systemic administration of a pharmaceutically acceptable imaging composition comprising biocompatible and stable nanoparticles that are fluorescent in the NIR range, more particularly, biocompatible and stable nanoparticles to which a NIR fluorescent probe, as the sole active agent, is non-covalently linked. Particular such particles exemplified herein are phospholipid particles in the form of either liposomes or micelles, wherein the fluorescent probe is adsorbed to or embedded within the particles; and inclusion complexes of the fluorescent probe and either an hydroxyalkyl- or sulphoalkylether-cyclodextrin. The term “biocompatible” as used herein with respect to the particles composing said imaging composition means that said particles are made of compounds suitable for administration to humans; and the term “stable” as used herein means that these particles are both physically and chemically stable, i.e., can be stored for a substantial period of time (e.g., weeks, months or years), and are not chemically degraded under physiological conditions for a period of time longer than about 30, 45, 60, 75, 90, 105 or 120 minutes.

The term “urinary pathways” as used herein refers to the urinary system also known as the “urinary tract”, consisting of the kidneys, ureters, the urinary bladder, and the urethra. Yet, the method of the present invention is mainly aimed at imaging the ureters, e.g., during an abdominal or pelvic surgery for intraoperative identification and prevention of ureteral injury.

In one aspect, the present invention relates to a method for imaging the urinary pathways, more particularly the ureters, of an individual in need thereof, said method comprising: (i) systemically administering to said individual an imaging composition containing a NIR fluorescent probe; and (ii) quantitatively or qualitatively measuring the emission intensity of said NIR fluorescent probe from the urinary pathways of said individual upon excitation at a proper wavelength, thereby imaging said urinary pathways, wherein said imagining composition comprises particles each independently comprising (a) a phospholipid, wherein said NIR fluorescent probe is non-covalently bound to said particle, i.e., either adsorbed to the outer surface of said particle or embedded within said particle; or (b) an inclusion complex of said NIR fluorescent probe and either an hydroxyalkyl- or sulphoalkylether-cyclodextrin.

In one particular such aspect, the present invention relates to a method as defined above, wherein the imaging composition administered comprises particles each comprising a phospholipid, i.e., liposomes or micelles, wherein said NIR fluorescent probe, as the sole active agent, is either adsorbed to or embedded within said particle.

In another particular such aspect, the present invention relates to a method as defined above, wherein the imaging composition administered comprises particles each comprising an inclusion complex of said NIR fluorescent probe and either an hydroxyalkyl-cyclodextrin or sulphoalkylether-cyclodextrin.

In a further particular such aspect, the present invention relates to a method as defined above, wherein the imaging composition administered comprises a combination of particles each independently comprising either (a) a phospholipid, wherein said NIR fluorescent probe, as the sole active agent, is adsorbed to or embedded within said particle; or (b) an inclusion complex of said NIR fluorescent probe and either an hydroxyalkyl-cyclodextrin or sulphoalkylether-cyclodextrin, provided that particles of both (a) and (b) are present.

The term “near infrared (NIR) fluorescent probe” as used herein refers to any fluorescent probe having an absorption and fluorescence spectrum in the NIR region. Examples of such fluorescent probes include, without being limited to, cyanine dyes such as indocyanine green (ICG), Cy5, Cy5.5, Cy5.18, Cy7 and Cy7.18; IRDye 78, IRDye 680, IRDye 750, IRDye 800 phosphoramidite (LI-COR Biosciences), DY-681, DY-731, DY-781 (Dyomics GmbH), or Alexa Fluor dyes such as Alexa Fluor® 610, Alexa Fluor® 633, Alexa Fluor® 647, Alexa Fluor® 660, Alexa Fluor® 680, Alexa Fluor® 700 and Alexa Fluor® 750. In particular embodiments, the NIR fluorescent probe contained within the imaging composition administered according to the method of the invention is ICG, which is currently the only US FDA-approved NIR molecule.

According to the method of the present invention, imaging of the urinary pathways of an individual is carried out by visualization of fluorescence and/or by measurement of the emission intensity of the NIR fluorescent probe from the urinary pathways of said individual, upon excitation at a proper wavelength, after systemic administration of the imaging composition which may be carried out as single-shot or repetitive administration. As defined above, the phrase “measuring the emission intensity of said NIR fluorescent probe” in step (ii) of said method refers to either quantitative measurement of the emission intensity of said fluorescent probe or qualitative measurement, i.e., detection, or said probe. The term “proper wavelength” with respect to the NIR fluorescent probe means any wavelength in the NIR region that would be suitable for excitation of the NIR fluorescent probe, and preferably the particular wavelength(s) at which the maximum emission intensity peak of the NIR fluorescent probe is observed.

In certain embodiments, the imaging composition administered according to the method of the present invention comprises a phospholipid-based particles, wherein said phospholipid is selected from a lecithin such as egg or soybean lecithin, or a derivative thereof, e.g., a lecithin having polyethylene glycol (PEG) chains; a phosphatidylcholine such as egg phosphatidylcholine; a hydrogenated phosphotidylcholine; a lysophosphatidylcholine; dipalmitoylphosphatidylcholine; distearoylphosphatidylcholine; dimyristoylphosphatidylcholine; dilauroylphosphatidylcholine; a glycerophospholipid such as phosphatidylglycerol, phosphatidylserine, phosphatidylethanolamine, lysophosphatidylethanolamine, phosphatidylinositol, phosphatidylinositol phosphate, phosphatidylinositol bisphosphate and phosphatidylinositol triphosphate; sphingomyelin; cardiolipin; a phosphatidic acid; a glycolipid such as a glyceroglycolipid, e.g., a galactolipid and a sulfolipid, a glycosphingolipid, e.g., a cerebroside (a glucocerebroside and a galactocerebroside), and a glycosylphosphatidylinositol; a plasmalogen; a phosphosphingolipid such as a ceramide phosphorylcholine, a ceramide phosphorylglycerol, and a ceramide phosphorylethanolamine; or a mixture thereof.

In particular non-limiting embodiments, the imaging composition administered according to the method of the invention comprises phospholipid-based particles, wherein said phospholipid is a commercially available product such as Phospholipon® 50, Phospholipon® 75, Phospholipon® 85G or Phospholipon® 90G, essentially consisting of soybean lecithins and phospholipids; Phospholipon® 80H or Phospholipon® 90H, essentially consisting of hydrogenated soybean lecithins and phospholipids; Phospholipon® E25, Phospholipon® E35 or Phospholipon® E, essentially consisting of egg yolk lecithins and phospholipids; and Phospholipon® LPC20, Phospholipon® LPC25 or Phospholipon® LPC65, essentially consisting of partially hydrolyzed soybean lecithins (all of Lipoid). In a more particular embodiment, the imaging composition comprises phospholipid-based particles, wherein said phospholipid is Phospholipon® 50, i.e., a soybean lecithin with about 45% phosphatidylcholine and about 10 to about 18% phosphatidylethanolamine, or Phospholipon® 75, i.e., a soybean lecithin with about 75% phosphatidylcholine.

In certain embodiments, the imaging composition administered according to the method of the present invention comprises phospholipid-based particles, wherein said phospholipid is admixed with one or more, e.g., two, three or four, nonphosphorous-containing molecules. Non-limiting examples of suitable nonphosphorous-containing molecules include fatty amines such as octylamine, laurylamine, N-tetradecylamine, hexadecylamine, stearylamine, oleylamine, tallowamine, hydrogenated tallowamine, and cocoamine; fatty acids; fatty acid amides; esters of fatty acid such as isopropyl myristate, hexadecyl stearate, and cetyl palmitate; cholesterol; cholesterol esters; diacylglycerols; or glycerol esters such as glycerol ricinoleate.

The phospholipid-based particles composing the imaging composition of the present invention are negatively charged and have zeta potential of ≧|10| mV (absolute value), and their size is preferably about 60 nm or less. In order to prevent uptake by the reticuloendothelial system and hence increase circulating time of the particles, a PEGylated phospholipid can be admixed with the phospholipid composing the particle.

In certain embodiments, the imaging composition administered according to the method of the present invention comprises a phospholipid-based particles, wherein said phospholipid is optionally admixed with one or more nonphosphorous-containing molecules as defined above, and thus further admixed with one or more, e.g., two, three or four, PEGylated phospholipids. Examples of suitable PEGylated phospholipids include, without being limited to, PEGylated dipalmitoyl phosphatidylethanolamine (DPPE-PEG), PEGylated palmitoyloleoyl phosphatidylethanolamine (POPE-PEG), PEGylated dioleoyl phosphatidylethanolamine (DOPE-PEG) and PEGylated distearoyl phosphatidylethanolamine (DSPE-PEG), preferably 1,2-distearoyl-sn-glycero-3-phosphoethanolamine-N-[polyethyleneglycol 2000] (PEG-DSPE-2000). In a particular embodiment, the imaging composition administered according to the method of the invention comprises phospholipid-based particles, wherein said phospholipid is optionally admixed with one or more nonphosphorous-containing molecules as defined above, and further admixed with PEG-DSPE-2000, wherein each one of the particles comprises up to 15% by weight of PEG-DSPE-2000.

In certain embodiments, the imaging composition administered according to the method of the present invention comprises a phospholipid-based particles, wherein said phospholipid is optionally admixed with one or more nonphosphorous-containing molecules and/or one or more PEGylated phospholipids as defined above, and each one of said particles comprises said NIR fluorescent probe in a weight ratio that enables the highest fluorescent signal after administration of said imaging composition, e.g., about 2%, 4%, 6%, 8%, 10%, 12%, 14%, 16%, 18%, 20% or more, by weight of said NIR fluorescent probe, depending of the actual performance.

In particular embodiments, the imaging composition administered according to the method of the invention comprises phospholipid-based particles, wherein said phospholipid is either Phospholipon® 50 or Phospholipon® 75, and each one of said particles comprises, e.g., 5% to 20% but preferably 10% to 20% by weight, ICG, either adsorbed to or embedded within said particle.

The term “inclusion complex” also referred to as “inclusion compound”, as used herein, refers to a complex in which one chemical compound (“the host”), in this particular case an hydroxyalkyl-cyclodextrin or a sulphoalkylether-cyclodextrin, forms a cavity in which molecules of a second chemical compound (“the guest”), in this particular case a NIR fluorescent probe, are located and actually trapped. There is no covalent bonding between guest and host, and the attractions are generally due to van der Waals forces.

Cyclodextrins (CDs) are a family of cyclic oligosaccharides composed of 5 or more α-D-glucopyranoside units linked 1→4, in the ⁴C₁ chair conformation. The most common cyclodextrins have six, seven or eight glucopyranose units and are referred to as α-, β- and γ-CD, respectively. As a consequence of their peculiar structure, these molecules feature a conical cavity that is essentially hydrophobic in nature and limited by hydroxyl groups of different chemical characters. The hydroxyl groups located at the narrower side are primary, i.e., come from position 6 of the glucopyranose ring, while those located at the wider entrance are secondary and therefore are less prone to chemical transformation. The reactivity of the hydroxyl groups strongly depends on the reaction conditions. The non-reducing character of cyclodextrins makes them behave as polyols. On the other hand, the large number of hydroxyl groups available implies that careful selection of the reaction conditions is required in order to avoid the substitution of more groups than those needed for a particular purpose.

The inner diameter of the conical cavity in unmodified cyclodextrins varies from 5 to 10 Å and its depth is about 8 Å. For β-CDs, the internal and external diameters are about 7.8 Å and 15.3 Å, respectively, and the calculated surface area is approximately 185 Å². These dimensions allow the inclusion of several types of guest molecules of appropriate size to form inclusion complexes. As a consequence of the inclusion, some properties of the guest molecules change, and this phenomenon, in fact, constitutes the basis of practically all cyclodextrin applications, including artificial enzymes, sensors, drug formulations, cosmetics, food technology and textiles. Cyclodextrin inclusion complexes can be thermodynamically stable depending on the shape and size of the guest molecule, and the association constants can be measured by a range of physicochemical methods. Absorption and emission spectroscopy along with nuclear magnetic resonance and calorimetry are the most popular techniques used to study these systems and have provided an understanding of the structure and energetics of the inclusion process. Recently, the use of scanning probe techniques such as atomic force microscopy has allowed the measurement of the force involved in these interactions at a single-molecule level, opening new and exciting prospects in supramolecular chemistry.

In certain embodiments, the imaging composition administered according to the method of the present invention comprises particles each independently comprising an inclusion complex of said NIR fluorescent probe and an hydroxyalkyl-cyclodextrin, wherein said hydroxyalkyl-cyclodextrin is hydroxyalkyl-α-, hydroxyalkyl-β- or hydroxyalkyl-γ-cyclodextrin, preferably hydroxyalkyl-β-cyclodextrin. The term “hydroxyalkyl” as used herein refers to any hydroxyl derivative of a C₁-C₄ alkyl, i.e., a straight or branched saturated hydrocarbon radical having 1-4 carbon atoms such as methyl, ethyl, n-propyl, isopropyl, n-butyl, sec-butyl, isobutyl and tert-butyl. In more particular embodiments, said hydroxyalkyl-β-cyclodextrin is hydroxyethyl-β-cyclodextrin, hydroxypropyl-β-cyclodextrin, dihydroxypropyl-β-cyclodextrin, or hydroxybutyl-β-cyclodextrin, preferably hydroxypropyl-β-cyclodextrin, more preferably HPβCD.

In certain embodiments, the imaging composition administered according to the method of the present invention comprises particles each independently comprising an inclusion complex of said NIR fluorescent probe and a sulphoalkylether-cyclodextrin, wherein said sulphoalkylether-cyclodextrin is sulphoalkylether-α-, sulphoalkylether-β- or sulphoalkylether-γ-cyclodextrin, preferably sulphoalkylether-β-cyclodextrin. The term “sulphoalkylether” as used herein refers to a group of the general formula —O—(C₁-C₄)alkylene-SO₃H, wherein (C₁-C₄)alkylene typically means a divalent straight or branched hydrocarbon radical having 1-4 carbon atoms such as methylene, ethylene, propylene, butylene and 2-methylpropylene. In more particular embodiments, said sulphoalkylether-β-cyclodextrin is sulphoethylether-β-cyclodextrin, sulphopropylether-β-cyclodextrin, sulphobutylether-β-cyclodextrin, or sulphopentylether-β-cyclodextrin, preferably Captisol®.

The hydroxyalkyl or sulphoalkylether (—O—(C₁-C₄)alkylene-SO₃H) groups are randomly substituted onto the hydroxyl groups of the cyclodextrin and the amount of substitution is called the average degree of substitution or number of hydroxyalkyl or sulphoalkylether groups per cyclodextrin, and it is the preferred manner of describing the substitution. The molecular weight of the hydroxyalkyl- or sulphoalkylether-cyclodextrin is calculated based upon the degree of substitution, wherein said substitution is, in fact, a distribution around the average degree of substitution of the number of hydroxyalkyl or sulphoalkylether groups per cyclodextrin molecule with some molecules having either more or less than the average degree of substitution. The result is a mixture of many molecular species with respect to the number and location of substitutions around the ring of the cyclodextrin.

The degree of substitution may have an effect on the binding of guests to the hydroxyalkyl- or sulphoalkylether-cyclodextrin molecule, wherein at low degrees of substitution, binding is very similar to that of the unmodified cyclodextrin, while increasing substitution can lead to weakened binding due to steric hindreance. The effect on the binding of guests to the host molecule is dependent upon the particular guest and it is also possible to obtain increased binding due to an increase in surface area to which the guest can bind. Still, with most guests, these differences in binding with degree of substitution are small if detectable.

In certain embodiments, the imaging composition administered according to the method of the present invention comprises particles each independently comprising an inclusion complex of said NIR fluorescent probe and an hydroxyalkyl- or sulphoalkylether-cyclodextrin as defined above, wherein said hydroxyalkyl- or sulphoalkylether-cyclodextrin may have any degree of substitution, i.e., may be either fully or partially modified with hydroxyalkyl or sulphoalkylether groups, wherein each α-D-glucopyranoside units has three hydroxyl groups which can be substituted. In certain embodiments, the hydroxyalkyl-cyclodextrin is an hydroxyalkyl-β-cyclodextrin, preferably HPβCD, having a degree of substitution in a range of 3 to 8, preferably 3.5 to 7; or the sulphoalkylether-cyclodextrin is an sulphoalkylether-β-cyclodextrin, preferably Captisol®, having a degree of substitution in a range of 3 to 8, preferably 3.5 to 7.

In certain particular embodiments, the imaging composition administered according to the method of the invention comprises particles each comprising an inclusion complex of ICG and HPβCD at any degree of substitution, e.g., in a range of 3 to 8, preferably 3.5 to 7.

In other particular embodiments, the imaging composition administered according to the method of the invention comprises particles each comprising an inclusion complex of ICG and Captisol® at any degree of substitution, e.g., in a range of 3 to 8, preferably 3.5 to 7.

The particles composing the imaging composition administered according to the method of the present invention, whether comprising (a) a phospholipid wherein a NIR fluorescent probe as the sole active component is non-covalently bound to said particle; or (b) an inclusion complex of said NIR fluorescent probe and either an hydroxyalkyl- or sulphoalkylether-cyclodextrin, are nanoparticles. The term “nanoparticles” as used herein refers to materials and structures or particles having a uniform shape, e.g., spherical or elongated, or a variety of shapes, wherein each particle has at least one dimension (such as width) which is a micron or smaller in size, e.g., in the range of 20-100 nanometers, but preferably in the range of 20-80 or 30-60 nm, although other dimensions (such as length) may be longer than a micron.

In certain embodiments, the imaging composition is systemically administered in step (i) of the method of the invention as single or repetitive administration, and concomitantly with the measuring and imaging step (ii).

Study 1 hereinafter shows the efficacy of a composition comprising phospholipid (liposomal)-based particles, each comprising either Phospholipon® 50 or Phospholipon® 75 wherein ICG is either adsorbed to or embedded within said particle, vs. a composition comprising free ICG, in imaging the ureters of FVB mice during a surgical procedure representing an abdominal or pelvic surgery. As particularly shown, after intravenous administration of the composition, systemic ICG accumulation did not differ between mice treated with liposomal ICG and those treated with the free compound. However, whole body imaging revealed enhanced ICG renal elimination in the liposomes-treated mice, as demonstrated by the increased urinary bladder emission intensity, and this finding was confirmed by ex vivo measurement of ICG emission, demonstrating greater signal in ureters from mice treated with liposomes, compared to controls. Similarly to the findings from the ex vivo analysis, liposomal ICG injection resulted in greater emission intensity when intact ureters were imaged in mice following surgical exposure. These findings are of particular interest, because while many liposomal formulations reduce the drug fraction excreted in the urine (mostly due to limited glomerular filtration of the liposomes), these liposomes surprisingly enhance ureters detectability by ICG, and it can thus advance visualization of the urinary tract and prevent urethral injury intraoperatively thus avoiding injury and reducing morbidity and medical costs.

Study 2 shows the efficacy of a composition comprising ICG-HPβCD inclusion complexes vs. a composition comprising free ICG, in imaging the ureters of FVB mice during a surgical procedure. As clearly shown, the ICG-HPβCD-based composition enabled specific labeling of the ureters while free dye did not, indicating that the ICG-HPβCD inclusion complex is renally excreted whereas free ICG is negligibly excreted in the urine.

Study 3 shows the preparation of Phospholipon® 75 (liposomal)-based particles by sonication using the Adaptive Focused Acoustics™ technology that enables controlling liposome size mainly by time of sonication (longer times of sonication led to smaller particle size), and the binding of ICG to the liposomes prepared. As shown, the fluorescence intensity of the liposomal solution was kept for 24 hours, and the fluorescence intensity of the lyophilized (non-dissolved) kept liposomes was longer (about 2 weeks) after dissolution. In order to determine the appropriate liposome size for optimal in vivo imaging, various sizes of liposomes were injected to FVB mice as described in Study 1, and as found, the optimal liposomal size for ureteral imaging was around 30-60 nm, and the highest signal was obtained using liposomes comprising 10%-20% by weight ICG.

The method of the invention, in any one of the embodiments defined above, can thus be used for improving the detectability thus imaging the urinary pathways, more particularly the ureters, of an individual during an abdominal or pelvic surgery, e.g., for intraoperative identification or prevention of iatrogenic ureteral injury, thus reducing morbidity and medical costs.

In another aspect, the present invention provides an imaging composition as disclosed above, i.e., an imaging composition comprising particles each independently comprising (a) a phospholipid as defined above, wherein a NIR fluorescent probe, as the sole active agent, is either adsorbed to or embedded within said particle; or (b) an inclusion complex of said NIR fluorescent probe and either an hydroxyalkyl- or sulphoalkylether-cyclodextrin as defined above, for use in imaging the urinary pathways, more particularly the ureters.

In a further aspect, the present invention relates to use of an imaging composition as defined above for the preparation of a medicament for imaging the urinary pathways, more particularly the ureters.

In certain embodiments, the imaging composition of the present invention is used for repetitively imaging the urinary pathways of an individual during an abdominal or pelvic surgery, e.g., for intraoperative identification or prevention of iatrogenic ureteral injury.

In certain embodiments, the imaging composition of the present invention comprises particles each comprising a phospholipid, i.e., liposomes or micelles, wherein said NIR fluorescent probe is either adsorbed to said particle or embedded within said particle. Particular such imaging compositions comprise particles each comprising either Phospholipon® 50 or Phospholipon® 75, wherein ICG is either adsorbed to or embedded within said particle.

In certain embodiments, the imaging compositions of the present invention comprises particles each comprising an inclusion complex of said NIR fluorescent probe and either an hydroxyalkyl-cyclodextrin or sulphoalkylether-cyclodextrin. Particular such imaging compositions comprise particles each comprising an inclusion complex of ICG and either HPβCD or Captisol®.

In a further particular such aspect, the imaging compositions of the present invention comprises a combination of particles each independently comprising either (a) a phospholipid, wherein said NIR fluorescent probe, as the sole active agent, is adsorbed to or embedded within said particle; or (b) an inclusion complex of said NIR fluorescent probe and either an hydroxyalkyl-cyclodextrin or sulphoalkylether-cyclodextrin, provided that particles of both (a) and (b) are present. Particular such imaging compositions comprise particles each comprising either (a) Phospholipon® 50 or Phospholipon® 75, wherein ICG is either adsorbed to or embedded within said particle; or (b) an inclusion complex of ICG and either HPβCD or Captisol®.

Phospholipid-based particles, e.g., liposomes or micelles, having a NIR fluorescent probe either adsorbed to or embedded within for use in the imaging composition of the present invention can be prepared according to any procedure and/or technique known in the art, e.g., as described in WO 2012/032524 and/or conducted herein. For example, small unilamellar liposomes can be prepared by high-energy sonication of a phospholipid or a mixture of phospholipids as defined herein, and a NIR fluorescent probe binding can then be performed by incubating the liposomes prepared with a solution of the fluorescent probe. The adsorbed quantity of the NIR fluorescent probe may be calculated by measuring the optical density of the solution obtained after filtering the sample to thereby remove all the liposomes. When preparing those liposomal particles, solutions containing various concentrations of the fluorescent probe might be used, aimed at preparing liposomal particles comprising as high concentration of the fluorescent probe as possible, without causing aggregation of the particles or decreasing the fluorescent signal.

Solutions containing cyclodextrin-based particles for use in the imaging composition of the present invention, i.e., particles each consisting of an inclusion complex of a NIR fluorescent probe and a hydroxyalkyl- or sulphoalkylrther-cyclodextrin, can be prepared according to any procedure and/or technique known in the art, e.g., as conducted herein, i.e., by mixing the fluorescent probe with the particular hydroxyalkyl- or sulphoalkylrther-cyclodextrin in a particular molar ratio in a suitable buffer.

The pharmaceutically acceptable imaging composition administered according to the method of the invention may be prepared by conventional techniques, e.g., as described in Remington: The Science and Practice of Pharmacy, 19^{th} Ed., 1995. The composition can be prepared, e.g., by uniformly and intimately bringing the active ingredient, i.e., the particles composing the imaging composition as defined above, into association with a liquid carrier. In certain embodiments, the imaging composition is liquid, and may further include pharmaceutically acceptable fillers, carriers, diluents or adjuvants, and other inert ingredients and excipients. In other embodiments, the imaging composition is in the form of a powder, which disperses well upon contact with an injectable liquid. Imaging compositions in the form of a powder can be prepared by any suitable method known in the art, e.g., by lyophilization (freeze drying) or spray drying.

The imaging composition administered according to the method of the invention can be formulated for any suitable parenteral route of administration, e.g., for intravenous, intraarterial, intramuscular, intraperitoneal, intrathecal or subcutaneous administration, but they are preferably formulated for intravenous administration. The imaging composition may be in the form of a sterile injectable aqueous solution or suspension, e.g., in a non-toxic parenterally acceptable diluent or solvent, and may be formulated according to the known art using suitable dispersing, wetting or suspending agents. Acceptable vehicles and solvents that may be employed include, without limiting, water, Ringer's solution and isotonic sodium chloride solution. The dosage administered as well as the duration and rate of administration will be determined as deemed appropriate by the practitioner.

The detection of NIR emission from the urinary pathways according to the method of the invention may be carried out utilizing any suitable means, i.e., an appropriate intraoperative NIR imaging system like, without being limited to, Mini-Fluorescence-Assisted Resection and Exploration (FLAIR)™ imaging system or a robotic system like the da Vinci™ surgical system (Intuitive Surgical).

The invention will now be illustrated by the following non-limiting Examples.

### EXAMPLES

### Study 1. Imaging Composition Comprising Phospholipid-Based (Liposomal) Particles

### Material and Methods

### Materials

ICG was purchased from Acros Organics (Geel, Belgium). Phospholipon® 50 and Phospholipon® 75 were obtained from Lipoid (Steinhausen, Switzerland). 1,2-Dimyristoyl-sn-glycero-3-phosphocholine (DMPC) was purchased from Avanti (Alabaster, USA). Pentobarbitone sodium was purchased from CTZ (Hod-Hasharon, Israel). All other reagents were from Sigma-Aldrich (Rehovot, Israel).

### Animals

The animal study protocol was approved by the Hebrew University Institutional Animal Care and Use Committee and the procedures followed were in accordance with institutional guidelines. Female FVB mice (7-8 weeks old) were purchased from Harlan Laboratories (Rehovot, Israel). The mice had free access to food (a standard diet) and water, and they were maintained on a 12/12-h automatically timed light/dark cycle.

### Liposome Preparation and ICG Binding

Liposomes from Phospholipon® 50 (Lipoid) were prepared as described in WO 2012/032524. In particular, 1 g of Phospholipon® 50 was dispersed by magnetic stirring in phosphate buffer (pH 7, 2 mM, sucrose 9.3% w/w). 60 mg of ICG (Acros) was solubilized in 12.5 ml of double distilled water (DDW). Liposome dispersion was mixed with ICG solution at ratio of 1:5 v/v overnight at 4° C.

Liposomes composed of Phospholipon® 50 were prepared by high-energy sonication which typically leads to formation of small unilamellar liposomes. Dynamic light scattering (DLS) measurements showed that the mean size (by volume) of the liposomes is 29.8±0.6 nm with a polydispersity index of 0.235-0.250, and zeta potential of −30.5±1 mV (at pH 7). Cryo-TEM (transmission electron microscopy) imaging confirmed that the liposomes are unilamellar in the size range of 20-40 nm, in agreement with DLS results.

The ICG binding experiments were performed by incubation of the liposomes with solutions of ICG at various concentrations. The adsorbed quantity of ICG was calculated by measuring the optical density of the solution obtained after filtration of the samples by a 300-kDa filter, which is expected to remove all the liposomes. The retained particles had a green color, giving a visual indication that the ICG is bound to the liposomes.

The quantity of adsorbed ICG increased with the increase in ICG concentration in solution. The liposome number was calculated according to Jones (2005). A bilayer thickness was taken as 4 nm, and a cross-sectional area of phospholipid as 0.71 nm² (Tone et al., 2007). It should be noted that high ICG concentrations caused aggregation of liposomes, and the maximal concentration of ICG that did not cause aggregation in this composition was 46 molecules per liposome. For the greatest ICG concentration in the adsorption isotherm (1519 molecules per liposome), the aggregate size, as seen by optical microscope, is 1.4-4.2 μm (data not shown). In addition to the size increase, it was found that binding ICG caused a decrease in the absolute value of zeta potential from −30.5±1 mV to −15.35±0.35 mV.

### Fluorescence of the ICG-Adsorbed Liposomes

The fluorescence spectra of the ICG-adsorbed liposomes prepared were evaluated in comparison to those of ICG in aqueous solution. As found, the fluorescence intensity increases with the increase of ICG concentration, reaches a maximum at 6.4×10⁻³ mM, and is followed by a gradual decrease. The concentration at which the maximum is observed is close to that previously reported (Saxena et al., 2003) (2.6×10⁻³ mM) for ICG in aqueous solution. As clearly shown, the fluorescence intensity is greater for liposomal ICG throughout nearly the entire concentration range studied.

The decrease in fluorescence intensity at high ICG concentrations, both in solution and in liposomal dispersion, was explained by quenching due to formation of aggregates of dye molecules (Saxena et al., 2003). The ICG emission peak in water was observed at 805 nm at concentrations less than 6.4×10⁻³ mM; above that there is a shift up to 814 nm. Those results correlate well with data previously reported (Saxena et al., 2003). The dependence of fluorescence in dispersions of liposomes with bound ICG is different: up to 3.2×10⁻³ mM ICG the emission peak occurs at about 820 nm, whereas above this concentration a red shift that is dependent on ICG concentration is observed. Increasing the ICG concentration to 9.6×10⁻² mM causes a shift to about 30 nm.

The red shift is accompanied with quenching of fluorescence, i.e., the larger the red shift the lower the fluorescence intensity. Because the fluorescence intensity of ICG-liposomes was greater than that of unbound ICG molecules (aqueous solution), the quantum yield ratio between the two states in which ICG is present was determined, for a concentration of 1.28×10⁻³ mM. ICG is poorly stable in aqueous solutions due to degradation, and therefore, in order to evaluate the stability of the ICG-adsorbed liposomes, the absorption of this fluorescent probe, when dissolved in an aqueous solution or adsorbed to the liposomes, was measured at light and at dark over time. As found, while dissolved ICG absorption decreases over time both in light and in dark, the absorption of ICG when bound to the liposomes almost does not change, and while light further decreases dissolved ICG absorption due to photobleaching, the absorption of ICG when bound to the liposomes remains the same, indicating that photobleaching is not significant in this case. Based on these findings, it can be concluded that the stability of ICG when bound to the liposomes is significantly increased compared with that of ICG dissolved in an aqueous solution.

The relative quantum yield was calculated according to the following equation (Fery-Forgues and Lavabre, 1999):

$$\frac{{φ}_{x}}{{φ}_{s}} = ( \frac{{A}_{s}}{{A}_{x}} ) \times ( \frac{{F}_{x}}{{F}_{s}} ) \times {( \frac{{n}_{x}}{{n}_{s}} )}^{2}$$

where φ is the quantum yield, F is the area under the emission peak (expressed in number of photons), A is absorbance at the excitation wavelength, and n is the refractive index of the solvents. The subscript x denotes the respective values of the sample (liposome-ICG), and s denotes standard (free ICG in buffer). As found, the quantum yield of liposomal ICG is more than three times greater than that of free ICG in buffer solution.

### Animal Studies

Mice were shaved under isoflurane (1-2%, v/v) anesthesia and underwent baseline scans using an IVIS Kinetic® in vivo imaging system (Caliper Life Sciences, Hopkinton, Mass., USA). Two hundred μl of 8 mg/kg Phospholipon® 50 ICG liposomes or the free compound in 2 mM sucrose buffer (phosphate buffer pH 7, with 9.3% w/w sucrose) were injected into the tail vein and the mice were repetitively scanned over a time period of up to 120 min.

For additional ex vivo studies, animals were treated as described above and sacrificed under anesthesia by cervical dislocation, 10 min post injection. Cardiac blood was collected in heparinized 96-well plates and tissues were harvested. Along the collection procedure, tissue and blood samples were protected from light and kept on ice. Immediately thereafter, blood and tissues were scanned by Typhoon FLA 9500 biomolecular imager (GE Healthcare Life Sciences, Piscataway Township, NJ, USA).

An additional cohort of animals was used for further studies of the accumulation of liposomal ICG in the urinary system. Liposomal or free ICG was injected into the tail vein, and 3 min later, mice were treated with high dose pentobarbital administered intraperitoneally. Overall time of active circulation after the ICG injection was about 10 min. Thereafter, mice were subjected to midline laparotomy with full exposure of the retroperitoneum. The ureters were identified in their origin from the kidneys, and distally clipped or loosely tied for identification. The mice were also hysterectomyzed to ease the urinary bladder identification. Subsequently, the animals were scanned by the Typhoon FLA 9500 biomolecular imager. Each experiment was repeated at least three times.

### Pyrene Fluorescence Measurements

0-0.8 μM ICG was incubated with 1% liposomes and 20 μM or 200 μM pyrene for evaluation of polarity and fluidity, respectively. Fluorescence spectra were measured by Carry Eclipse fluorescence spectrophotometer (Varian Medical Systems; Palo Alto, Calif., USA) at excitation wavelength 335 nm.

### Differential Scanning Calorimetric (DSC) Studies

DMPC (1 g) was solubilized in 10 ml chloroform. The solvent was evaporated and the film was hydrated in 5 mM phosphate buffer (pH 7). The dispersion was sonicated by a UP200S Hielscher (Teltow, Germany) sonicator probe for 10 min. ICG aqueous solution was added to the liposomes and the samples were incubated overnight at 4° C. The thermal behavior of the samples was measured by Mattler Toledo (Greifensee, Switzerland) DSC 822. The temperature range was 5-45° C. and the heating rate was 1° C./min.

### Data Analysis

Analysis of in vivo ICG pharmacokinetics (PK), including region of interest (ROI) placement (Living Image; PerkinElmer, Waltham, Mass., USA; (Schneider et al., 2012)) and calculation of the area under the concentration-time curve (AUC) from the time of injection to the time of the last measurement (Phoenix WinNonlin; Certara; St. Louis, Mo., USA), was conducted as previously described (Portnoy et al., 2012). Partial areas were calculated for the AUC between the time of ICG injection (0 min) and 60 or 120 min after injection (AUC_{0-60 min} and AUC_{0-120 mm}, respectively). Data are presented as means±SD. The non-parametric Mann-Whitney or Kruskal-Wallis tests, as appropriate, were used to determine the statistical significance of difference (p<0.05) between experimental groups (InStat; GraphPad, La Jolla, Calif., USA).

### Results

### Whole Body NIR Imaging

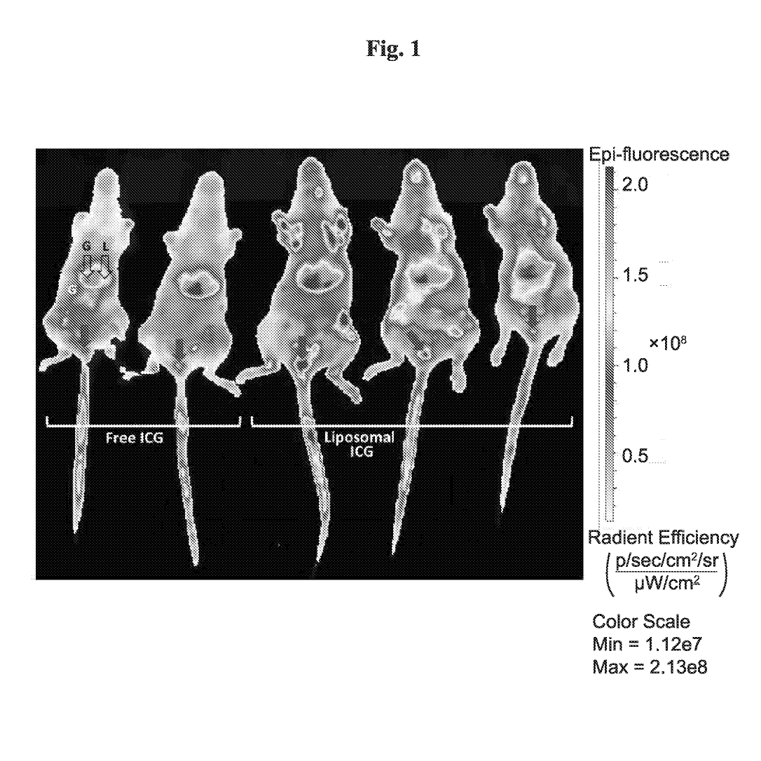

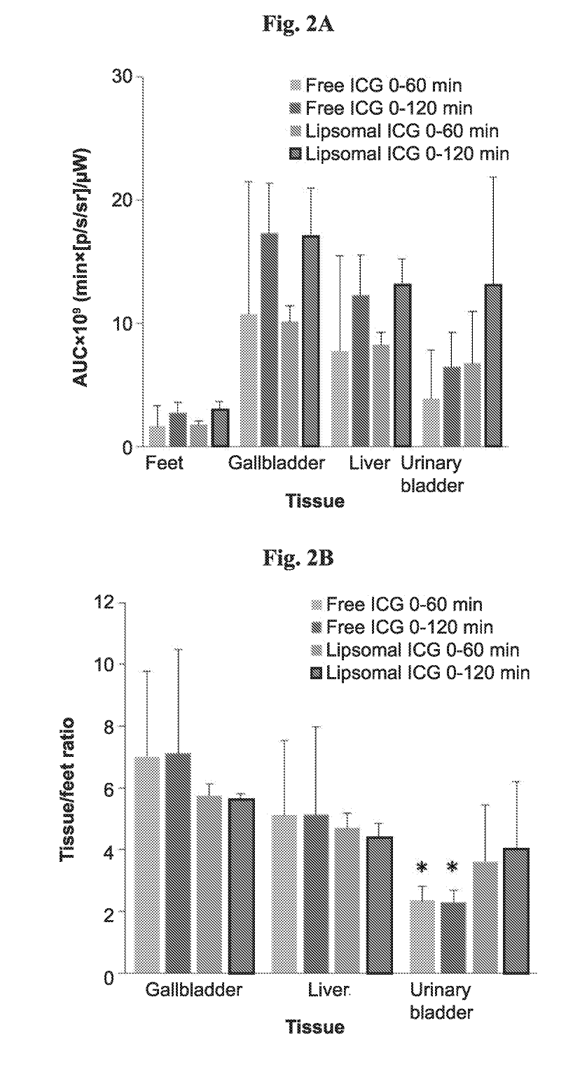

Following intravenous administration, both free and liposomal ICG accumulated in the liver and in the gallbladder (FIG. 1). The urinary bladder could be clearly observed and emission was particularly high in most of the liposome-treated mice. Unfortunately, the in vivo imaging system did not allow us to detect the kidneys even when the animals were positioned with their backs facing the camera. Overall, the cumulative in vivo emission intensity of the urinary bladder in liposomes-treated mice was similar to that in the liver and the gallbladder (FIG. 2A). In these animals, the urinary bladder/feet AUC_{0-60 min} and AUC_{0-120 min} ratios of liposomes-treated mice were 3.6±1.8 and 4.0±2.2, respectively, not significantly different from those of the gallbladder (FIG. 2B). In contrast, in control mice, the emission intensity AUC of the urinary bladder was only 2.3 fold that of the feet. Hence, the feet-normalized urinary bladder AUC was significantly lower than that of the gallbladder (2.3±0.5 vs. 7.0±2.8 for AUC_{0-60 min} and 2.3±0.4 vs. 7.1±3.3 for AUC_{0-120 min}, respectively; P<0.05 for both comparisons).

### Ex-Vivo Imaging

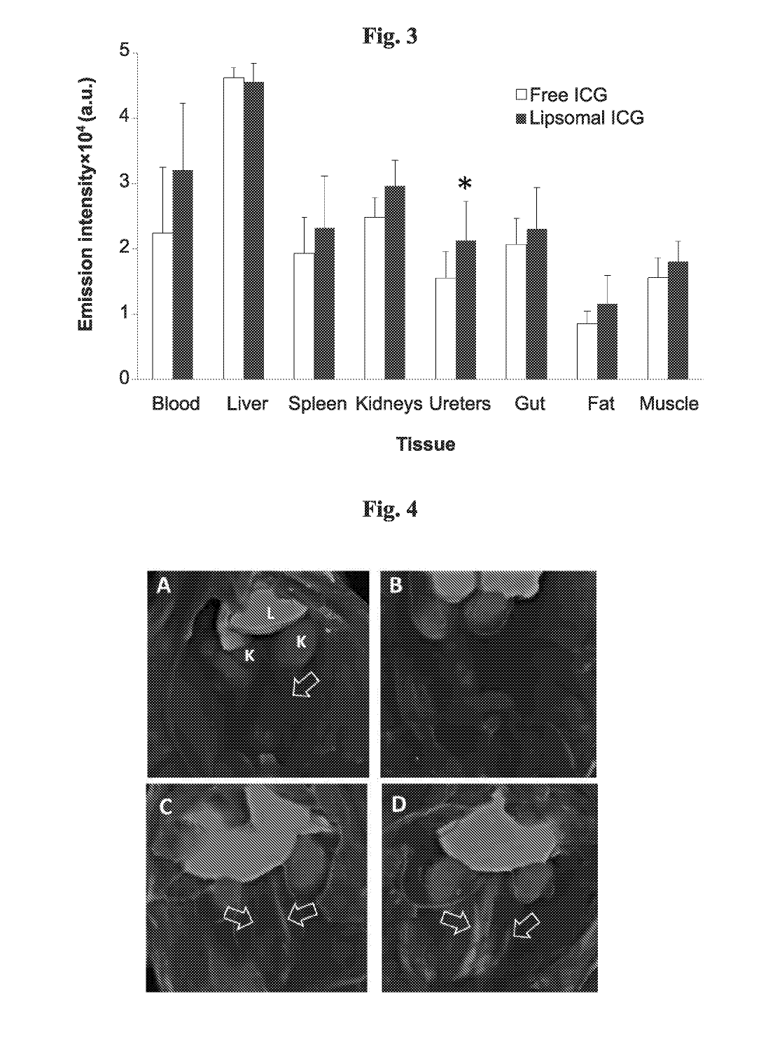

Ten min after intravenous injection, both free and liposomal ICG accumulated mostly in the liver, the kidney and the spleen (FIG. 3). A significant difference between the two ICG formulations was observed only in ureteral emission intensity, which was 40% greater (P<0.05) in the liposomal vs. free ICG.

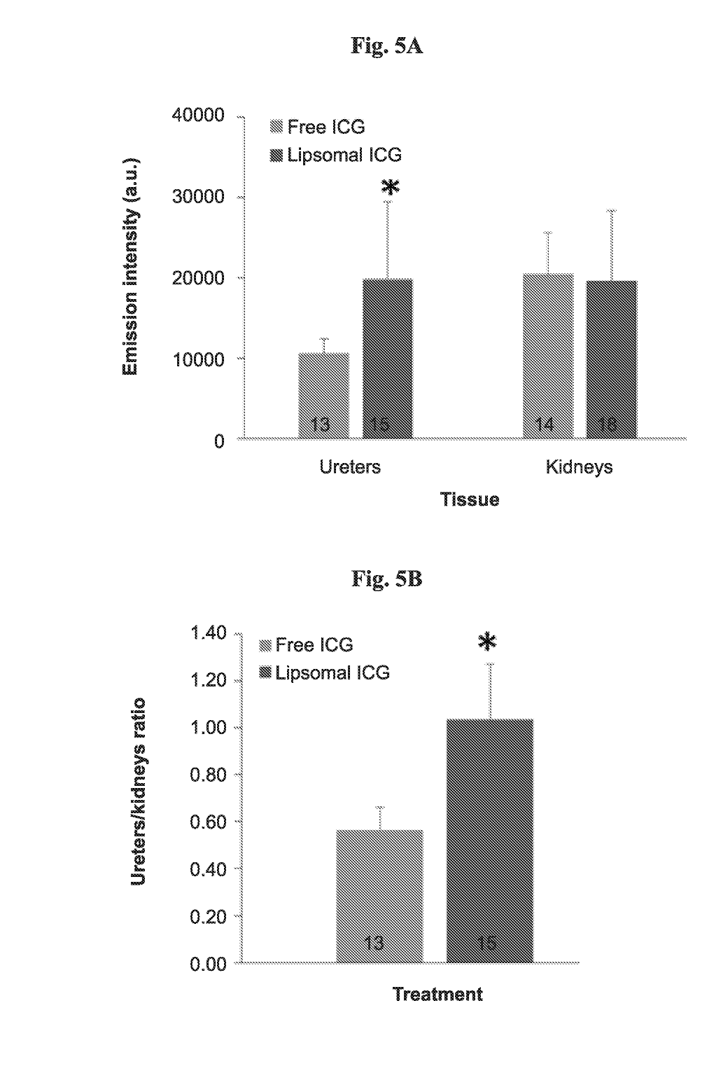

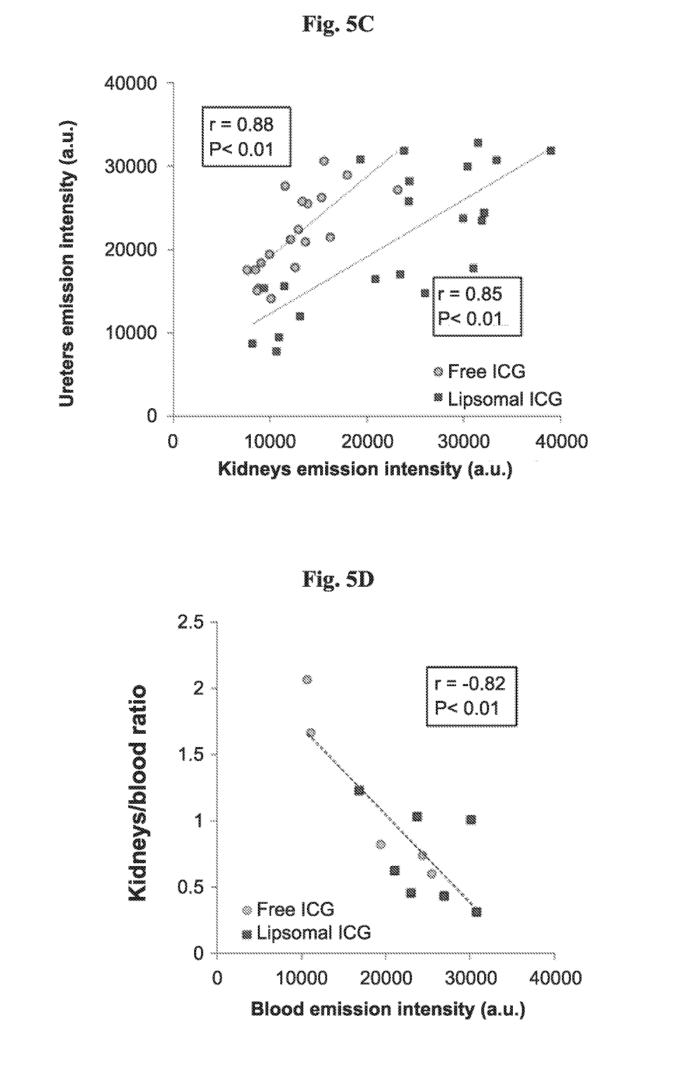

To further explore the potential of liposomal ICG as a fluorescent probe for surgical imaging of the urinary tract, the kidneys and the ureters were scanned following their surgical exposure in anesthetized female mice. Administration of liposomal ICG significantly (1.9-fold, P<0.01) enhanced the ureteral, but not the renal emission intensity (FIG. 4 and FIG. 5A). The differences in the emission ratio remained significant after normalization of ureteral emission to renal emission (1.03±0.24 vs. 0.56±0.10 for liposomal and free ICG, respectively, P<0.01; FIG. 5B). For both ICG formulations, ureteral ICG emission correlated with that of the kidneys (FIG. 5C). In a combined analysis of both treatment groups, the kidneys/blood ratio correlated inversely to blood emission intensity (r=−0.82; P<0.01; FIG. 5D). Ureters/blood intensity also correlated to blood intensity, although to a lesser extent (r=−0.56, P=0.057; data not shown).

ICG Interaction and Localization within Liposomes

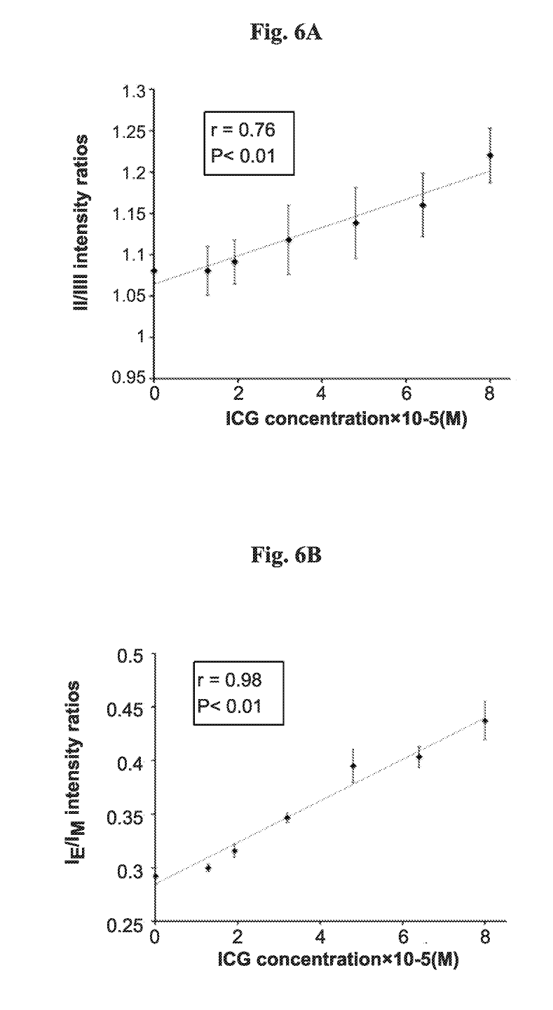

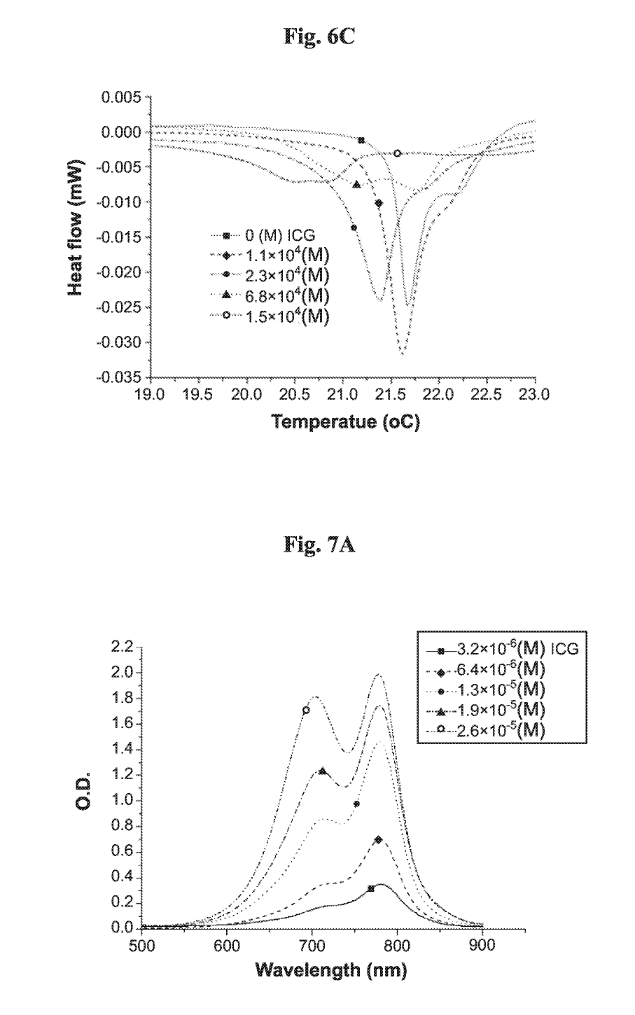

To better understand the basis for the altered PK of ICG passively adsorbed to liposomes, the localization of ICG within the liposome was characterized. Pyrene fluorescence spectra measurements were used to examine the effects of ICG on lipid bilayer organization. A typical monomer spectrum of pyrene is characterized by five vibrationic bands between 360-420 nm (Ioffe and Gorbenko, 2005) and structureless spectrum of excimer (pyrene's excited state dimer (Rotermund et al., 1997)) with peak at 475 nm. The analysis demonstrated that greater ICG concentrations were associated with increases in both micropolarity (the intensity ratios of peak I; at 375 nm) to peak III; at 384 nm; r=0.76; FIG. 6A) and microviscosity (the excimer-to-monomer intensity ratio I_{E}/I_{M}; 480 nm and 384 nm, respectively; r=0.98; FIG. 6B).

An additional indication that the ICG is embedded within the lipid bilayer was provided by measurement of thermal transitions of DMPC. Increasing the ICG concentration induced changes in the phase behavior of membranes, as reflected by broadening the peak and shifting it to a lower temperatures range (FIG. 6C).

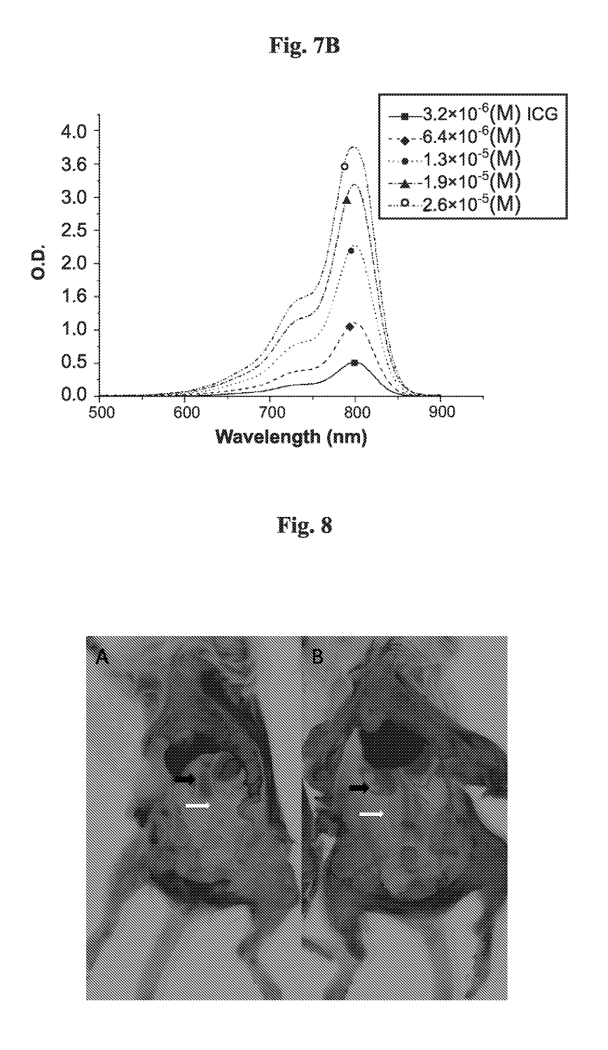

Absorbance spectra also revealed that ICG was present in the bilayer predominantly as its monomeric form (at 780 nm) while peaks of weakly florescent dimers (702-730) (Philip et al., 1996) were observed for ICG in solution (FIG. 7).

### DISCUSSION

Our aim was to evaluate liposomal ICG as a tool for ureteral imaging. Previous studies demonstrated that ICG encapsulation in liposomes and polymeric nanoparticles significantly increases its plasma concentrations and alters its tissue distribution (see, e.g., Kraft and Ho, 2014; Ma et al., 2012; Saxena et al., 2006; Yaseen et al., 2007; Zheng et al., 2012). In the current study we focused on ICG renal excretion, assuming that liposomal encapsulation may help shift ICG elimination from liver to kidneys. Even small increases in ICG transfer into ureters can increase their detectability, because normally, renal ICG elimination after systemic administration is negligible (Bax et al., 1980; Cherrick et al., 1960).

### Imaging Studies

In vivo, systemic ICG accumulation did not differ between mice treated with liposomal ICG and those treated with the free compound (FIGS. 1-2). However, whole body imaging revealed enhanced ICG renal elimination in the liposomes-treated mice, as demonstrated by the increased urinary bladder emission intensity (FIG. 2). This finding was confirmed by ex vivo measurement of ICG emission, which demonstrated greater signal in ureters from mice treated with liposomes, compared to controls (FIG. 3). In the same animals, compared to free ICG, liposomal ICG emission in blood was 1.4-fold greater. However, this difference did not achieve statistical significance. Likewise, the slightly greater emission of liposomal ICG in other tissues was not statistically significant. Similarly to the findings from the ex vivo analysis, liposomal ICG injection resulted in greater emission intensity when intact ureters were imaged in mice following surgical exposure (FIGS. 4-5). These findings are of particular interest, because many liposomal formulations, such as Doxil (Gabizon et al., 1994) and AmBisomem (Bekersky et al., 2002) reduce the drug fraction excreted in the urine, mostly due to limited glomerular filtration of the liposomes. Surprisingly, we found that the liposomes enhance ureters detectability by ICG: In fact, enhanced renal elimination is a merit of the ICG liposomal formulation, because it can advance visualization of the urinary tract and prevent urethral injury intraoperatively. To overcome ICG relatively rapid elimination its injection may be repeated if necessary.

The exact mechanism that governs enhanced ureteral ICG emission is unclear. It is likely that the ICG signal obtained from the ureters reflects the free compound and not the liposomes. This is because the diameter of the liposomes is 30 nm, well above the threshold for glomerular filtration (approximately 6-8 nm) (Ishida et al., 2002; Longmire et al., 2008). Alternatively, the liposomal preparation may slow down the ICG renal delivery and potentially prevent saturation of renal transport. This would allow renal elimination of a greater fraction of the injected ICG dose. Overcoming saturation of low-capacity renal transporters is supported by the negative correlation between kidney/blood emission intensity ratio and blood emission intensity (FIG. 5D), as well as by the increased ureters/kidney emission intensity ratio upon administration of liposomal ICG (FIG. 5B). Whereas the ureters/kidneys emission intensity ratio was 0.5 for free ICG, it doubled for the liposomal formulation (FIG. 5B). Of note, the kidney signal combines emission from blood, urine and renal tissue, and the signal from urine (and blood) may contribute to the overall renal signal. This assumption is based on the clear correlation between ureteral and renal ICG emission (r=0.88 and 0.85 for free and liposomal ICG, respectively; FIG. 4, panel C).

ICG Interaction and Location within Liposomes

Pyrene is a highly hydrophobic probe, located in the lipid bilayer liposomes (Mishra et al., 2000). Measurement of its emission spectra is used for examination of molecules effects on lipid bilayer organization. Therefore, the positive correlation between increased I_{I}/I_{III} ratio and ICG concentration (r=0.76, P<0.01); FIG. 6A) is suggestive of enhanced membrane polarity due to the presence of ICG (Dao Dong, 1984). This may be explained by migration of pyrene to outer regions of the liposome (L′Heureux and Fragata, 1988) or by liposomal higher water permeability due to structural changes (Sexana et al., 2003). The I_{I}/I_{III} values 1.08-1.22 obtained in our measurements are in line with previously reported values for similar systems, which do not contain ICG: 0.9-1.2 for dipalmitoylphosphatidylcholine (DPPC) and 1.1 for soybean phosphatidylcholine (Ioffe and Gorbenko, 2005). The fluidity of lipids (as measured by the microviscosity ratio I_{E}/I_{M}, 480 nm and 384 nm, respectively; FIG. 6B) may be estimated by the excimer formation (Dao Dong, 1984), which is assumed to be a diffusion-dependent process (Ioffe and Gorbenko, 2005; Engelke et al., 1995). Therefore, increased excimer-to-monomer intensity ratio with increasing ICG concentration (r=0.98; P<0.01), which is ICG concentration-dependent, further supports the assumption that ICG is incorporated into the lipid bilayer as its major distribution site within liposomes.

Differential scanning colorimetry (DSC) is an additional established method for investigation of interactions of molecules with liposomes (Aburai et al., 2013; El Maghraby et al., 2005). Thermal transitions in the presence of ICG were measured for DMPC liposomes. The main phase transition likely resulted from melting of alkyl chains in the hydrophobic part from a rigid gel phase to a fluid liquid crystalline phase (Barry et al., 2009). Increasing ICG concentration modified the DMPC thermograms through peak broadening and decreased the area as well as their shift to a lower temperatures range (FIG. 6C). Those changes indicate that ICG altered the lipid structures, possibly due to interference with inter-chain hydrophobic bonds, suggesting that ICG is located in the hydrophobic part of the bilayer (Pedroso de Lima et al., 1990; Yoneya and Noyori, 1993). These results are in line with those obtained through the pyrene fluorescence measurements as well as with previously reported changes in fluorescence and absorbance spectra associated with hydrophobic environments (WO 2012/032524; Philip et al., 1996; Proulx et al., 2010) and accumulation of ICG within polymeric micells hydrophobic core (Proulx et al., 2010).

Within the bilayer, ICG is presented predominantly as monomers (FIG. 7), while in solution ICG formed dimers, even at very low concentration ˜3×10⁸), with consequent reduced quantum yield (Saxena et al., 2003).

### Study Limitations

This study was designed to evaluate liposomal ICG imaging properties in vivo and not to be a PK study aimed at estimating ICG clearance. Prior to this study it was hypothesized that incorporation of ICG in the liposome would increase the visualization of the urinary tract, and the study was therefore focused on analysis of the signal from this pathway and not on the fraction of the dose excreted in urine. Nevertheless, the enhanced signal from ureters and the initial findings from the in vivo imaging suggest that the liposomal formulation may increase ICG urinary excretion. Because ICG half live in plasma is in the minutes range (Bax et al., 1980; Cherrick et al., 1960), ICG emission was measured on an early time point after ICG injection, to optimize its detection. In addition, this time frame is appropriate for its intended use-intraoperative imaging. The presumed scenario would be injection of the ICG-liposome upon discretion of the surgeon, when the ureters should be identified during the operation. We assume that ureteral secretion of the dye would occur in minutes. Alternatively, modification of the liposomal formulation to attenuate ICG release may allow a single liposomal injection per surgery.

Because the ICG is bound to the liposomes, there could be disadvantages for application of such system. In particular, ICG from the liposomal bilayer may be released to the plasma upon dilution and bind to plasma proteins as a function its membrane/medium partition coefficient (Barenholz, 2012). However, the main advantage of this system is that all components are FDA approved for medical application, and there is no chemical modification of the ICG. Overall, the results presented herein provide a proof-of-concept for the ability of simple nano-formulations to enable ureter detectability using ICG as the fluorescent imaging probe during surgeries.

### CONCLUSIONS

The altered PK of liposomal ICG enables imaging of ureters following systemic administration of the formulation. Because the excretion of free ICG into urine is minimal, even slight increase in its urinary elimination may have significant impact on its emission intensity in the urinary pathways. This property could be utilized for ureteral imaging intraoperatively, thus avoiding injury and reducing morbidity and medical costs.

Liposomes from Phospholipon® 75 were prepared using the same procedures described above with respect to Phospholipon® 50, and the various experiments described in this Study were repeated using those liposomes, showing similar results.

### Study 2. Imaging Composition Comprising Cyclodextrin-Based Particles

As stated above, HPβCD and Captisol® are FDA approved for various administration routes and known to be eliminated mostly by renal excretion. In this study, inclusion complexes of HPβCD with ICG were used so as to increase the fraction of ICG eliminated through the kidney to thereby specifically labeling the ureters.

Sample Preparation.

60 mg of ICG (Acros) was solubilized in 12.5 ml of DDW. 2 ml of ICG solution were added to 10 ml of phosphate buffer (pH 7, 2 mM, and sucrose 9.3% w/w). 18.5 mg of HPβCD (Acros) were added to ICG buffered solution and mixed for 10 h at 4° C. Free ICG 8 mg/kg in phosphate-sucrose buffer, or the corresponding cyclodextrin complex solution (200 μl), was injected to female FVB mice (7-8 weeks old). Mice were sacrificed 10 min post injection and imaged by Typhoon imager (General Electric).

### Results

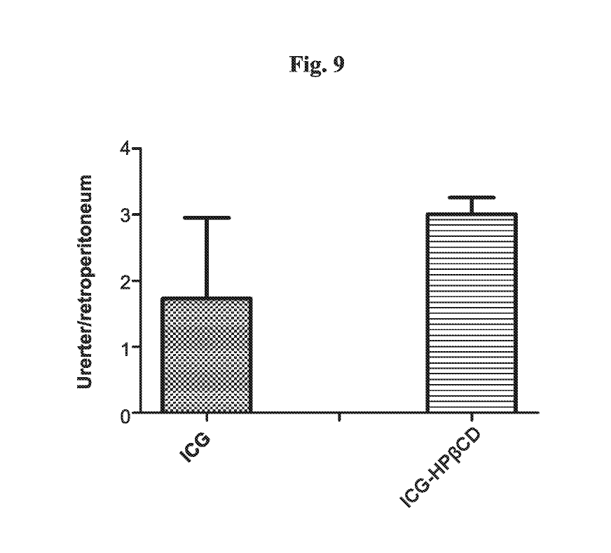

As found, ICG in cyclodextrin solution specifically labelled ureters while free dye did not. FIG. 8 shows a representative experiment out of 3 experiments conducted, indicating that the inclusion complex of ICG and HPβCD is renally excreted, whereas free ICG is negligibly excreted in the urine. FIGS. 8A and 8B show FVB mice injected with free ICG and ICG-HPβCD, respectively, wherein the black arrow shows the kidney, and the white arrow shows the ureter. FIG. 9 shows the quantification of ICG-HPβCD complex brightness intensity in the ureter, as compared to the retroperitoneum that is the reference tissue for ureteral identification. The results summarize 3 different experiments (n=6 for each group).

### Study 3. Imaging Composition Comprising Phospholipid-Based (Liposomal) Particles

### Liposome Preparation and ICG Binding

Liposomes from Phospholipon® 75 (Lipoid) were prepared by sonication using the Adaptive Focused Acoustics™ technology, which delivers controlled energy precisely and accurately to a sample tube while maintaining temperature control, and therefore enables controlling liposome size mainly by time of sonication. In particular, Phospholipon® 75 5% was dispersed in 2 mM phosphate buffer with 9.3% sucrose and stirred using a magnetic stirrer at room temperature for 40 minutes or until all solid material dissolved. All preparations were performed under Argon. 60-nm liposomes were then prepared by sonication using S220 Focused-ultrasonicators (Covaris M-series miniTubes) under the following conditions: peak incident power=40W, duty factor=50%, cycles per burst=1000, duration=480 seconds, bath temperature=20° C., power mode=frequency sweeping, degassing mode=N/A, volume=1 ml. 100-nm, 30-nm and <20-nm liposomes were prepared by the same procedure, wherein the duration parameter is changed to 180, 1,200 and 2,400 seconds, respectively.

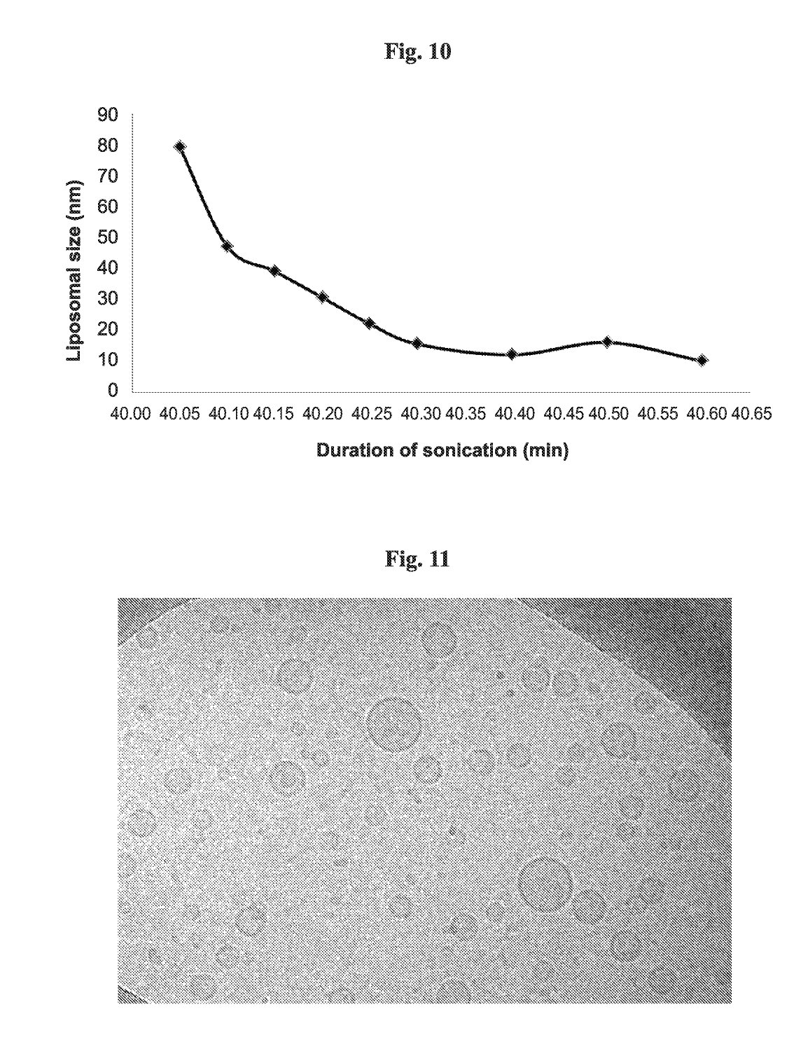

Liposome size measurements were performed using a Zetasizer Nano-S (Malvern Instruments, Worcestershire, United Kingdom). The aqueous dispersions were measured after dilution to 0.005%. Generally, longer times of sonication led to smaller particle size (FIG. 10).

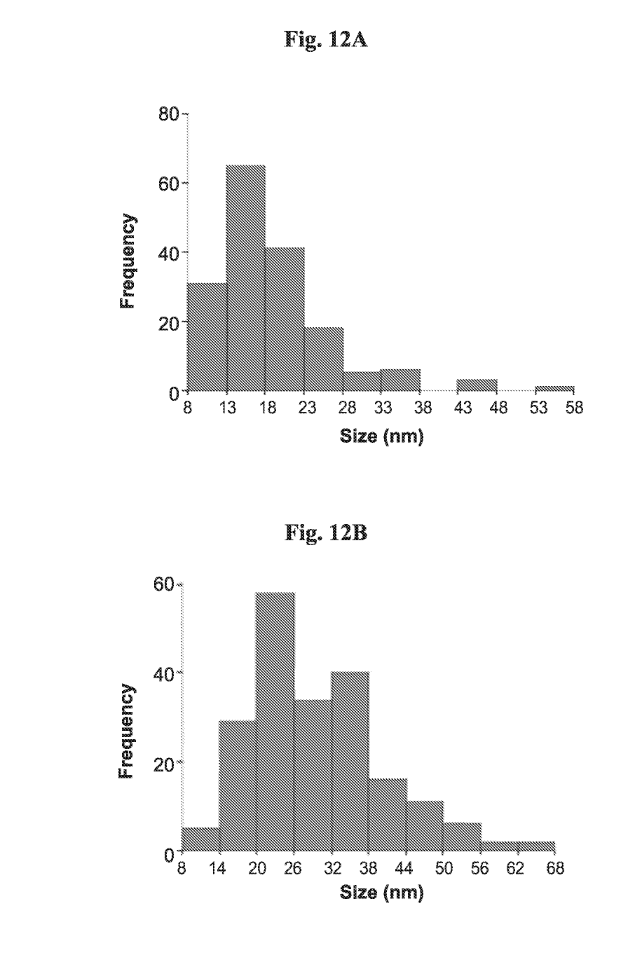

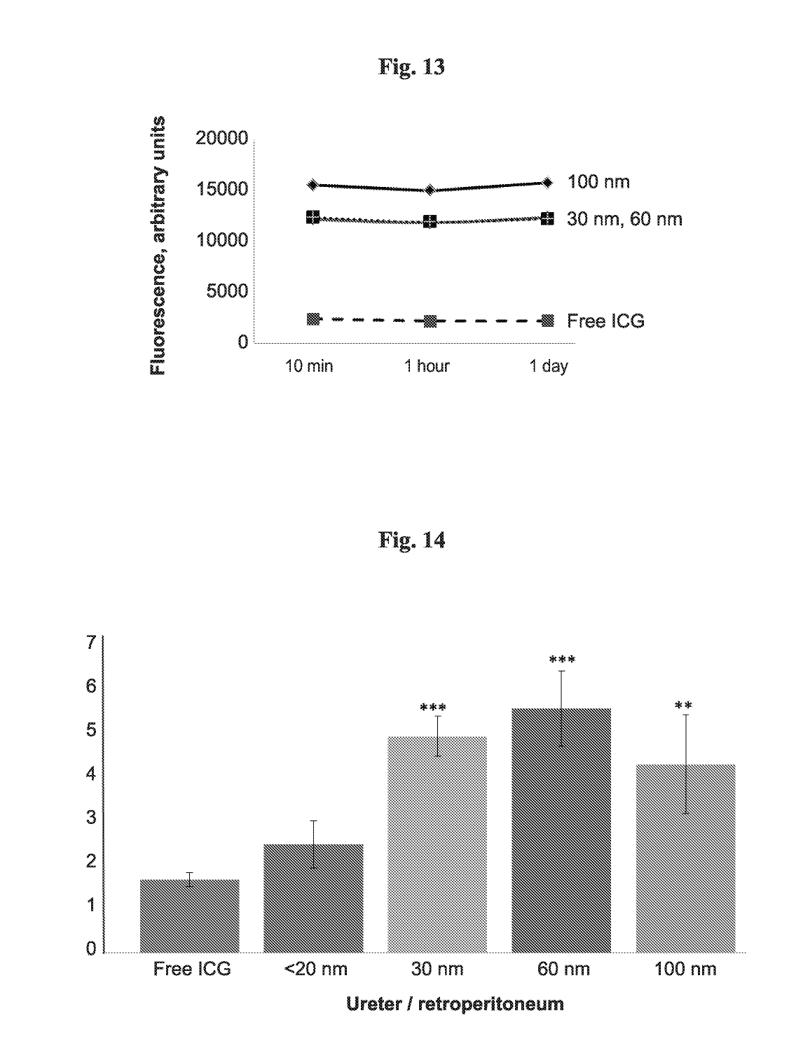

The liposome samples were diluted with phosphate buffer to 1% and the nanoparticles were then imaged using cryo-transmission electron microscopy (TEM) as previously describes (portnoy et al., 2011). The cryo-TEM microscopy of the particles showed spherical liposomes (FIG. 11) with size distribution as expected (FIGS. 12A-12B).

ICG (Acros Organics, Geel, Belgium) was dissolved in DDW to 3.2 mM ICG stock solution, and binding to liposomes was performed by adding ICG stock solution to liposomes in a ratio of 1:5 (ICG:liposomes). The dispersion was then incubated under mild agitation at 5° C. for 24 hours in the dark.

### Liposome Stability

Liposomal ICG stability was examined after dissolution in PBS. Fluorescence intensity was determined after different time points, while the liposomal solution was kept in the dark overnight at 4° C. As found, the fluorescence intensity was kept for 24 hours. Of note, if the lyophilized liposomes are not dissolved, their stability is longer (about 2 weeks).

### Optimal Liposomal Size and ICG Concentration

In order to determine the appropriate liposome size for optimal in vivo imaging, liposomes were prepared in various sizes. The animal study protocol was identical to that described in Study 1. FIG. 14 shows the effect of liposomal size on ureter visibility, indicating that the optimal liposomal size for ureteral imaging is around 30-60 nm. As further found, the highest fluorescent signal was obtained when liposome comprising 10-20% ICG (of total liposome weight) were used (data not shown).

### REFERENCES

Aburai K., Ogura T., Hyodo R., Sakai H., Abe M., Glatter O., Location of cholesterol in liposomes by using small-angle X-ray scattering (SAXS) data and the generalized indirect Fourier transformation (GIFT) method. J Oleo Sci., 2013, 62, 913-918
Assimos D. G., Patterson L. C., Taylor C., Changing incidence and etiology of iatrogenic ureteral injuries. J Urol., 1994, 152, 2240-2246
Barenholz Y., Doxil®—the first FDA-approved nano-drug: lessons learned. J Control Release, 2012, 160, 117-134
Barros T. C., Toma S. H., Toma H. E., Bastos E. L., Baptista M. S., Polymethine cyanine dyes in β-cyclodextrin solution: multiple equilibria and chemical oxidation. Journal of Physical Organic Chemistry, 2010, 23, 893-903
Barry J., Fritz M., Brender J. R., Smith P. E., Lee D. K., Ramamoorthy A., Determining the effects of lipophilic drugs on membrane structure by solid-state NMR spectroscopy: the case of the antioxidant curcumin. J Am Chem Soc., 2009, 131, 4490-4498
Bax N. D., Tucker G. T., Woods H. F., Lignocaine and indocyanine green kinetics in patients following myocardial infarction. Br J Clin Pharmacol., 1980, 10, 353-361
Bekersky I., Fielding R. M., Dressler D. E., Lee J. W., Buell D. N., Walsh T. J., Pharmacokinetics, excretion, and mass balance of liposomal amphotericin B (AmBisome) and amphotericin B deoxycholate in humans. Antimicrob Agents Chemother., 2002, 46, 828-833
Cherrick G. R., Stein S. W., Leevy C. M., Davidson C. S., Indocyanine green: observations on its physical properties, plasma decay, and hepatic extraction. J Clin Invest., 1960, 39, 592-600
Dao Dong M. W., The Py scale of solvent polarities. Can J Chem., 1984, 62, 2560-2565
Dzurinko V. L., Gurwood A. S., Price J. R., Intravenous and indocyanine green angiography. Optometry, 2004, 75, 743-755
El Maghraby G. M., Williams A. C., Barry B. W., Drug interaction and location in liposomes: correlation with polar surface areas. Int J Pharm., 2005, 292, 179-185
Engelke M., Klockmann H. C., Diehl H. A., Gramicidin affects the transverse and lateral distribution of pyrene and pyrene derived probes in lipid bilayers. Spectrochimica Acta Part A., 1995, 52, 85-91
Fery-Forgues S., Lavabre D., Are fluorescence quantum yields so tricky to measure? A demonstration using familiar stationery products. J Chem Educ, 1999, 76, 1260-1264
Gabizon A., Catane R., Uziely B., Kaufman B., Safra T., Cohen R., Martin F., Huang A., Barenholz Y., Prolonged circulation time and enhanced accumulation in malignant exudates of doxorubicin encapsulated in polyethylene-glycol coated liposomes. Cancer Res., 1994, 54, 987-992
Gould S, Scott R. C., 2-Hydroxypropyl-beta-cyclodextrin (HP-beta-CD): a toxicology review. Food Chem Toxicol., 2005, 43, 1451-1459
Huang L., Vore M., Multidrug resistance p-glycoprotein 2 is essential for the biliary excretion of indocyanine green. Drug Metab Dispos., 2001, 29, 634-637
Ioffe V., Gorbenko G. P., Lysozyme effect on structural state of model membranes as revealed by pyrene excimerization studies. Biophys Chem., 2005, 114, 199-204
Ishida T., Harashima H., Kiwada H., Liposome clearance. Biosci Rep., 2002, 22, 197-224
Jones M. N., Liposomal antibacterial, antifungal and antiviral agents. In: Nejan D, editor. Methods in enzymology, 2005, Vol. 391, p. 218. San Diego: Elsevier-Academic
Kraft J. C., Ho R. J., Interactions of indocyanine green and lipid in enhancing near-infrared fluorescence properties: the basis for near-infrared imaging in vivo. Biochemistry, 2014, 53, 1275-1283
Larach S. W., Gallagher J. T., Complications of laparoscopic surgery for rectal cancer: avoidance and management. Semin Surg Oncol., 2000, 18, 265-268
L'Heureux G. P., Fragata M., Micropolarities of lipid bilayers and micelles. 5. Localization of pyrene in small unilamellar phosphatidylcholine vesicles. Biophys Chem., 1988, 30, 293-301
Longmire M., Choyke P. L., Kobayashi H., Clearance properties of nano-sized particles and molecules as imaging agents: considerations and caveats. Nanomedicine (Lond)., 2008, 3, 703-717
Luo S., Zhang E., Su Y., Cheng T., Shi C., A review of NIR dyes in cancer targeting and imaging. Biomaterials, 2011, 32, 7127-7138
Ma Y., Sadoqi M., Shao J., Biodistribution of indocyanine green-loaded nanoparticles with surface modifications of PEG and folic acid. Int J Pharm., 2012, 436, 25-31
Meirow D., Moriel E. Z., Zilberman M., Farkas M., Evaluation and treatment of iatrogenic ureteral injuries during obstetric and gynecologic operations for nonmalignant conditions. J Am Coll Surg., 1994, 178, 144-148
Mishra A., Behera R. K., Behera P. K., Mishra B. K., Behera G. B., Cyanines during the 1990s: A Review. Chem Rev., 2000, 100, 1973-2012
Pedroso de Lima M. C., Chiche B. H., Debs R. J., Duzgunes N., Interaction of antimycobacterial and anti-pneumocystis drugs with phospholipid membranes. Chem Phys Lipids., 1990, 53, 361-371
Philip R., Penzkofer A., Baumler W., Szeimies R. M., Abels C., Absorption and fluorescence spectroscopic investigation of indocyanine green. J Photoch Photobio A., 1996, 96(1-3), 137-148
Polom W., Markuszewski M., Rho Y. S., Matuszewski M., Use of invisible near infrared light fluorescence with indocyanine green and methylene blue in urology. Part 2. Cent European J Urol., 2014, 67, 310-313
Portnoy E., Lecht S., Lazarovici P., Danino D., Magdassi S., Cetuximab-labeled liposomes containing near-infrared probe for in vivo imaging. Nanomedicine, 2011, 7(4), 480-488
Portnoy E., Gurina M., Magdassi S., Eyal S., Evaluation of the Near Infrared Compound Indocyanine Green as a Probe Substrate of P-Glycoprotein. Mol Pharm., 2012, 9, 3595-3601
Proulx S. T., Luciani P., Derzsi S., Rinderknecht M., Mumprecht V., Leroux J. C., Detmar M., Quantitative imaging of lymphatic function with liposomal indocyanine green. Cancer Res, 2010, 70, 7053-7062
Redan J. A., McCarus S. D., Protect the ureters. JSLS., 2009, 13, 139-141
Rotermund F., Weigand R., Holzer W., Wittmann M., Penzkofer A., Fluorescence spectroscopic analysis of indocyanine green J aggregates in water. J Photochem Photobiol A Chem, 1997, 110, 75-78
Sandanaraj B. S., Gremlich H. U., Kneuer R., Dawson J., Wacha S., Fluorescent nanoprobes as a biomarker for increased vascular permeability: implications in diagnosis and treatment of cancer and inflammation. Bioconjug Chem, 2010, 21, 93-101
Saxena V., Sadoqi M., Shao J., Degradation kinetics of indocyanine green in aqueous solution. J Pharm Sci., 2003, 92(10), 2090-2097
Saxena V., Sadoqi M., Shao J., Polymeric nanoparticulate delivery system for Indocyanine green: biodistribution in healthy mice. Int J Pharm., 2006, 308, 200-204
Schneider C. A., Rasband W. S., Eliceiri K. W., NIH Image to ImageJ: 25 years of image analysis. Nature Methods, 2012, 9, 671-675
Sorinola O., Begum R., Prevention and management of ureteric injuries. Hosp Med., 2005, 66, 329-34
Stella V. J., He Q., Cyclodextrins. Toxicol Pathol., 2008, 36, 30-42
Still J. M., Law E. J., Klavuhn K. G., Island T. C., Holtz J. Z., Diagnosis of burn depth using laser-induced indocyanine green fluorescence: a preliminary clinical trial. Burns, 2001, 27, 364-371
Summerton D. J., Kitrey N. D., Lumen N., Serafetinidis E., Djakovic N., European Association of Urology. EAU guidelines on iatrogenic trauma. Eur Urol., 2012, 62, 628-639
Tanaka E., Ohnishi S., Laurence R. G., Choi H. S., Humblet V., Frangioni J. V., Real-time intraoperative ureteral guidance using invisible near-infrared fluorescence. J Urol., 2007, 178, 2197-2202
Tone L. G., Carneiro A. L., Rosada R. S., Silva C. L., Santana M. H. A., A mathematical model describing the kinetic of cationic liposome production from dried lipid films adsorbed in a multitubular system. Braz J Chem Eng, 2007, 24, 477-486
Troyan S. L., Kianzad V., Gibbs-Strauss S. L., Gioux S., Matsui A., Oketokoun R., Ngo L., Khamene A., Azar F., Frangioni J. V., The FLARE intraoperative near-infrared fluorescence imaging system: a first-in-human clinical trial in breast cancer sentinel lymph node mapping. Ann Surg Oncol., 2009, 16, 2943-2952
Vakili B., Chesson R. R., Kyle B. L., Shobeiri S. A., Echols K. T., Gist R., Zheng Y. T., Nolan T. E., The incidence of urinary tract injury during hysterectomy: a prospective analysis based on universal cystoscopy. Am J Obstet Gynecol., 2005, 192, 1599-1604
Yaseen M. A., Yu J., Wong M. S., Anvari B., Stability assessment of indocyanine green within dextran-coated mesocapsules by absorbance spectroscopy. J Biomed Opt., 2007, 12, 064031
Yoneya S., Noyori K., Improved visualization of the choroidal circulation with indocyanine green angiography. Arch Ophthalmol., 1993, 111, 1165-1166
Zheng C., Zheng M., Gong P., Jia D., Zhang P., Shi B., Sheng Z., Ma Y., Cai L., Indocyanine green-loaded biodegradable tumor targeting nanoprobes for in vitro and in vivo imaging. Biomaterials, 2012, 33, 5603-5609

1-27. (canceled)

28. A method for imaging the urinary pathways of an individual in need thereof, said method comprising:
(i) systemically administering to said individual an imaging composition according to comprising particles each independently comprising (a) a phospholipid, wherein a near infrared (NIR) fluorescent probe is either adsorbed to or embedded within said particle; or (b) an inclusion complex of said NIR fluorescent probe and either an hydroxyalkyl- or sulphoalkylether-cyclodextrin, wherein each one of said particles has at least one dimension in the range of 30-60 nm; and
(ii) quantitatively or qualitatively measuring the emission intensity of said NIR fluorescent probe from the urinary pathways of said individual upon excitation at a proper wavelength, thereby imaging said urinary pathways.

29. The method of claim 28, wherein said imaging composition comprises particles each comprising Phospholipon® 50 or Phospholipon® 75 wherein ICG is either adsorbed to or embedded within said particle.

30. The method of claim 28, wherein said imaging composition comprises particles each comprising an inclusion complex of ICG and either 2-hydroxypropyl-β-cyclodextrin or sulphobutylether-β-cyclodextrin.

31. The method of claim 28, wherein said imaging composition is administered in step (i) as single or repetitive administration, and concomitantly with step (ii).

32. The method of claim 28, for single-shot or repetitive imaging of the urinary pathways of said individual during an abdominal or pelvic surgery.

33. The method of claim 32, for intraoperative identification or prevention of iatrogenic ureteral injury.

34. (canceled)

35. The method of claim 28, wherein said imaging composition comprises particles each comprising said phospholipid, wherein said NIR fluorescent probe is either adsorbed to or embedded within said particle.

36. The method of claim 28, wherein said imaging composition comprises particles each comprising an inclusion complex of a said NIR fluorescent probe and said hydroxyalkyl- or sulphoalkylether-cyclodextrin.

37. The method of claim 28, wherein said imaging composition comprises a combination of particles each independently according to (a) or (b), provided that particles of both (a) and (b) are present.

38. The method of claim 28, wherein said NIR fluorescent probe is a cyanine dye, IRDye 78, IRDye 680, IRDye 750, IRDye 800 phosphoramidite, DY-681, DY-731, DY-781, or an Alexa Fluor dye.

39. The method of claim 38, wherein said cyanine dye is indocyanine green (ICG), Cy5, Cy5.5, Cy5.18, Cy7, or Cy7.18; or said Alexa Fluor dye is Alexa Fluor 610, Alexa Fluor 633, Alexa Fluor 647, Alexa Fluor 660, Alexa Fluor 680, Alexa Fluor 700, or Alexa Fluor 750.

40. The method of claim 39, wherein said NIR, fluorescent probe is ICG.

41. The method of claim 28, wherein said phospholipid is a lecithin or a PEGylated derivative thereof, a phosphatidylcholine, a hydrogenated phosphotidylcholine, a lysophosphatidylcholine; dipalmitoylphosphatidylcholine, distearoylphosphatidylcholine, dimyristoylphosphatidylcholine, dilauroylphosphatidylcholine, a glycerophospholipid; sphingomyelin; cardiolipin, a phosphatidic acid, a glycolipid, a plasmalogen, a phosphosphingolipid, or a mixture thereof.

42. The method of claim 41, wherein said lecithin is egg lecithin, soybean lecithin, or a PEGylated derivative thereof; said phosphatidylcholine is egg phosphatidylcholin; said glycerophospholipid is phosphatidylglycerol, phosphatidylserine, phosphatidylethanolamine, lysophosphatidylethanolamine, phosphatidylinositol, phosphatidylinositol phosphate, phosphatidylinositol bisphosphate, or phosphatidylinositol triphosphate; said glycolipid is glyceroglycolipid, a glycosphingolipid, or a glycosylphosphatidylinositol; or said phosphosphingolipid is a ceramide phosphorylcholine, a ceramide phosphorylglycerol, or a ceramide phosphorylethanolamine.

43. The method of claim 42, wherein said glyceroglycolipid is a galactolipid, or a sulfolipid, or said glycosphingolipid is a cerebroside (a glucocerebroside and a galactocerebroside).

44. The method of claim 28, wherein said phospholipid is admixed with one or more nonphosphorous-containing molecules each independently is a fatty amine, a fatty acid, a fatty acid amide, an ester of a fatty acid, cholesterol, a cholesterol ester, a diacylglycerol, or a glycerol ester.

45. The method of claim 44, wherein said fatty amine is octylamine, laurylamine, N-tetradecylamine, hexadecylamine, stearylamine, oleylamine, tallowamine, hydrogenated tallowamine, or cocoamine; said ester of a fatty acid is isopropyl myristate, hexadecyl stearate, or cetyl palmitate; or said glycerol ester is glycerol ricinoleate.

46. The method of claim 28, wherein said phospholipid is admixed with one or more PEGylated phospholipids.

47. The method of claim 46, wherein said PEGylated phospholipid is PEGylated dipalmitoyl phosphatidylethanolamine (DPPE-PEG), PEGylated palmitoyloleoyl phosphatidylethanolamine (POPE-PEG), PEGylated dioleoyl phosphatidylethanolamine (DOPE-PEG), or PEGylated distearoyl phosphatidylethanolamine (DSPE-PEG), preferably 1,2-distearoyl-sn-glycero-3-phosphoethanolamine-N-[polyethyleneglycol 2000] (DSPE-PEG-2000).

48. The method of claim 28, wherein said hydroxyalkyl-cyclodextrin is hydroxyalkyl-α-, β- or γ-cyclodextrin.

49. The method of claim 48, wherein said hydroxyalkyl-cyclodextrin is hydroxyalkyl-β-cyclodextrin.

50. The method of claim 48, wherein said hydroxyalkyl-β-cyclodextrin is hydroxyethyl-β-cyclodextrin, hydroxypropyl-β-cyclodextrin, dihydroxypropyl-β-cyclodextrin, or hydroxybutyl-β-cyclodextrin.

51. The method of claim 50, wherein said hydroxyalkyl-β-cyclodextrin is 2-hydroxypropyl-β-cyclodextrin (HPβCD).

52. The method, of claim 28, wherein said sulphoalkylether-cyclodextrin is sulphoalkylether-α-, β- or γ-cyclodextrin.

53. The method of claim 52, wherein said sulphoalkylether-cyclodextrin is sulphoalkylether-β-cyclodextrin.

54. The method of claim 52, wherein said sulphoalkylether-β-cyclodextrin is sulphoethylether-β-cyclodextrin, sulphopropylether-β-cyclodextrin, sulphobutylether-β-cyclodextrin, or sulphopentylether-β-cyclodextrin.

55. The method of claim 54, wherein said sulphoalkylether-β-cyclodextrin is sulphobutylether-β-cyclodextrin (Captisol®).

56. The method of claim 29, wherein said particles comprise 10%-20% by weight ICG.
